(19) United States
(12) Patent Application Publication
(10) Pub. No.: US 2005/0079474 A1
(43) Pub. Date: Apr. 14, 2005

(54) Emotional state modification method and system

(76) Inventor: Kenneth Lowe, Wylie, TX (US)

Correspondence Address: Thomas P. Liniak;Liniak, Berenato & White, Suite 240, 6550 Rock Spring Drive, Bethesda, MD, 20817, US

(21) Appl. No.: 10/797,184

(22) Filed: Mar. 11, 2004

Related U.S. Application Data
(60) Provisional application No. 60/511,035, filed on Oct. 14, 2003.

(51) Int. Cl. G09B 19/00; G01N 33/569

(52) U.S. Cl. 434/236

(57) ABSTRACT

A method of categorizing electronically recallable media, in which a particular user is presented with media samples and permitted to provide personal responses to the media samples. From the personal responses, a determination is made whether the media samples evoke one or more of emotional states in the particular user. The media samples are electronically assigned to one or more of electronically recallable sets corresponding to the emotional states evoked in the particular user by the media samples. An aspect of the method allows for the selective recall of the media samples based on selection of one or more of the electronically recallable sets.

### RELATED APPLICATION

This application claims the benefit of priority of U.S. Provisional Patent Application Ser. No. 60/511,035 filed on Oct. 14, 2003 and entitled “COMPUTER AIDED MODIFICATION OF A GENERAL BEHAVIOR VIA SHAPING AND REINFORCEMENT,” the complete disclosure of which is incorporated herein by reference.

### COMPUTER PROGRAM LISTING

A computer program listing appendix is submitted herewith on compact disc recordable (CD-R) as Appendix A. Duplicate copies of Appendix A are provided as Copy 1 and Copy 2. The materials on the CD-R are identical to each other.

The files on the compact discs are incorporated herein by reference, and are listed below:

| | Size in | |
|---|---|---|
| File Name | bytes | Date |
| Affirmation10Page.cpp | 1,720 | 3/9/04 |
| Affirmation10Page.h | 575 | 3/9/04 |
| Affirmation20Page.cpp | 3,154 | 3/9/04 |
| Affirmation20Page.h | 718 | 3/9/04 |
| Affirmation30Page.cpp | 5,660 | 3/9/04 |
| Affirmation30Page.h | 845 | 3/9/04 |
| Affirmation40Page.cpp | 2,209 | 3/9/04 |
| Affirmation40Page.h | 606 | 3/9/04 |
| Affirmation50Page.cpp | 1,864 | 3/9/04 |
| Affirmation50Page.h | 605 | 3/9/04 |
| Affirmation60Page.cpp | 1,866 | 3/9/04 |
| Affirmation60Page.h | 608 | 3/9/04 |
| AffirmationSheet.cpp | 2,536 | 3/9/04 |
| AffirmationSheet.h | 1,277 | 3/9/04 |
| AffMaintenance.cpp | 1,577 | 3/9/04 |
| AffMaintenance.h | 781 | 3/9/04 |
| ChildDialog.cpp | 1,415 | 3/9/04 |
| ChildDialog.h | 727 | 3/9/04 |
| DefinedGoalsRpt.htm | 548 | 3/9/04 |
| DlgAffInstruct.cpp | 995 | 3/9/04 |
| DlgAffInstruct.h | 476 | 3/9/04 |
| DlgAffMaintenance.cpp | 12,082 | 3/9/04 |
| DlgAffMaintenance.h | 1,555 | 3/9/04 |
| DlgChangePassword.cpp | 3,143 | 3/9/04 |
| DlgChangePassword.h | 777 | 3/9/04 |
| DlgContemplation.cpp | 6,990 | 3/9/04 |
| DlgContemplation.h | 1,116 | 3/9/04 |
| DlgEditAffDesc.cpp | 2,400 | 3/9/04 |
| DlgEditAffDesc.h | 861 | 3/9/04 |
| DlgEnterAffirmations.cpp | 26,609 | 3/9/04 |
| DlgEnterAffirmations.h | 2,652 | 3/9/04 |
| DlgEnterAffirmations.htm | 2,022 | 3/9/04 |
| DlgGoalMaintenance.cpp | 11,665 | 3/9/04 |
| DlgGoalMaintenance.h | 1,605 | 3/9/04 |
| DlgHTMLAbout.cpp | 7,041 | 3/9/04 |
| DlgHTMLAbout.h | 735 | 3/9/04 |
| DlgHTMLAbout.htm | 1,153 | 3/9/04 |
| DlgListEntry.cpp | 1,764 | 3/9/04 |
| DlgListEntry.h | 785 | 3/9/04 |
| DlgLogEntry.cpp | 2,838 | 3/9/04 |
| DlgLogEntry.h | 1,116 | 3/9/04 |
| DlgReportCenter.cpp | 3,124 | 3/9/04 |
| DlgReportCenter.h | 867 | 3/9/04 |
| DlgTableReporter.cpp | 5,860 | 3/9/04 |
| DlgTableReporter.h | 1,852 | 3/9/04 |
| DlgTableReporter.htm | 2,098 | 3/9/04 |
| DlgTableReporter_CompletedAffirmations.cpp | 6,625 | 3/9/04 |
| DlgTableReporter_CompletedAffirmations.h | 630 | 3/9/04 |
| DlgTableReporter_CompletedGoals.cpp | 5,102 | 3/9/04 |
| DlgTableReporter_CompletedGoals.h | 523 | 3/9/04 |
| DlgTableReporter_DailyExercises.cpp | 5,885 | 3/9/04 |
| DlgTableReporter_DailyExercises.h | 635 | 3/9/04 |
| DlgTableReporter_DaysPracticed.cpp | 6,309 | 3/9/04 |
| DlgTableReporter_DaysPracticed.h | 695 | 3/9/04 |
| DlgTableReporter_DefinedAffirmations.cpp | 6,770 | 3/9/04 |
| DlgTableReporter_DefinedAffirmations.h | 622 | 3/9/04 |
| DlgTableReporter_DefinedGoals.cpp | 5,339 | 3/9/04 |
| DlgTableReporter_DefinedGoals.h | 517 | 3/9/04 |
| DlgTableReporter_FeedbackLogByDate.cpp | 4,058 | 3/9/04 |
| DlgTableReporter_FeedbackLogByDate.h | 600 | 3/9/04 |
| DlgTableReporter_FeedbackLogByGoal.cpp | 2,279 | 3/9/04 |
| DlgTableReporter_FeedbackLogByGoal.h | 355 | 3/9/04 |
| DlgTableReporter_ProgressLogByDate.cpp | 4,058 | 3/9/04 |
| DlgTableReporter_ProgressLogByDate.h | 600 | 3/9/04 |
| DlgTableReporter_ProgressLogByGoal.cpp | 2,279 | 3/9/04 |
| DlgTableReporter_ProgressLogByGoal.h | 355 | 3/9/04 |
| DlgTableReporter_SelfDescription.cpp | 15,314 | 3/9/04 |
| DlgTableReporter_SelfDescription.h | 943 | 3/9/04 |
| dsAffirmation.cpp | 8,020 | 3/9/04 |
| dsAffirmation.h | 2,074 | 3/9/04 |
| dsExerciseLog.cpp | 15,324 | 3/9/04 |
| dsExerciseLog.h | 3,010 | 3/9/04 |
| dsGoal.cpp | 9,323 | 3/9/04 |
| dsGoal.h | 2,113 | 3/9/04 |
| dsImage.cpp | 9,132 | 3/9/04 |
| dsImage.h | 2,083 | 3/9/04 |
| dsImportantRelationships.cpp | 2,965 | 3/9/04 |
| dsImportantRelationships.h | 1,001 | 3/9/04 |
| dsLog.cpp | 7,501 | 3/9/04 |
| dsLog.h | 1,860 | 3/9/04 |
| dsSelfDesc.cpp | 9,049 | 3/9/04 |
| dsSelfDesc.h | 1,597 | 3/9/04 |
| dsSession.cpp | 12,773 | 3/9/04 |
| dsSession.h | 1,863 | 3/9/04 |
| dsSlideShow.cpp_{—} | 5,706 | 3/9/04 |
| dsSlideShow.h_{—} | 1,437 | 3/9/04 |
| Exercise10.Page.cpp | 1,668 | 3/9/04 |
| Exercise10.Page.h | 557 | 3/9/04 |
| Exercise20.Page.cpp | 3,143 | 3/9/04 |
| Exercise20.Page.h | 659 | 3/9/04 |
| Exercise30.Page.cpp | 2,646 | 3/9/04 |
| Exercise30.Page.h | 662 | 3/9/04 |
| Exercise40.Page.cpp | 2,163 | 3/9/04 |
| Exercise40.Page.h | 591 | 3/9/04 |
| Exercise50.Page.cpp | 1,819 | 3/9/04 |
| Exercise50.Page.h | 590 | 3/9/04 |
| ExerciseSheet.cpp | 2,455 | 3/9/04 |
| ExerciseSheet.h | 1,161 | 3/9/04 |
| GettingStarted10Page.cpp | 1,878 | 3/9/04 |
| GettingStarted10Page.h | 627 | 3/9/04 |
| GettingStarted20Page.cpp | 2,127 | 3/9/04 |
| GettingStarted20Page.h | 650 | 3/9/04 |
| GettingStarted30Page.cpp | 1,955 | 3/9/04 |
| GettingStarted30Page.h | 652 | 3/9/04 |
| GettingStarted40Page.cpp | 1,904 | 3/9/04 |
| GettingStarted40Page.h | 627 | 3/9/04 |
| GettingStarted50Page.cpp | 1,857 | 3/9/04 |
| GettingStarted50Page.h | 626 | 3/9/04 |
| GettingStartedSheet.cpp | 2,631 | 3/9/ |
| GettingStartedSheet.h | 1,293 | 3/9/04 |
| GoalDef10Page.cpp | 2,013 | 3/9/04 |
| GoalDef10Page.h | 616 | 3/9/04 |
| GoalDef20Page.cpp | 4,789 | 3/9/04 |
| GoalDef20Page.h | 807 | 3/9/04 |
| GoalDef30Page.cpp | 2,082 | 3/9/04 |
| GoalDef30Page.h | 614 | 3/9/04 |
| GoalDef40Page.cpp | 7,891 | 3/9/04 |
| GoalDef40Page.h | 869 | 3/9/04 |
| GoalDef50Page.cpp | 4,355 | 3/9/04 |
| GoalDef50Page.h | 804 | 3/9/04 |
| GoalDef60Page.cpp | 2,209 | 3/9/04 |
| GoalDef60Page.h | 647 | 3/9/04 |
| GoalDefSheet.cpp | 2,402 | 3/9/04 |
| GoalDefSheet.h | 1,204 | 3/9/04 |
| GoalEntryDlg.cpp | 1,567 | 3/9/04 |
| GoalEntryDlg.h | 633 | 3/9/04 |
| GoalsFormView.cpp | 66,946 | 3/9/04 |
| GoalsFormView.h | 3,584 | 3/9/04 |
| GoalsOverviewView.cpp | 6,674 | 3/9/04 |
| GoalsOverviewView.h | 786 | 3/9/04 |
| HelpPropertySheet.cpp | 613 | 3/9/04 |
| HelpPropertySheet.h | 268 | 3/9/04 |
| Horizons.aps | 1,423,896 | 3/9/04 |
| Horizons.cpp | 17,958 | 3/9/04 |
| Horizons.h | 1,077 | 3/9/04 |
| Horizons.ncb | 2,296,832 | 3/9/04 |
| Horizons.rc | 101,275 | 3/9/04 |
| Horizons.reg | 712 | 3/9/04 |
| Horizons.sln | 916 | 3/9/04 |
| Horizons.vcproj | 34,447 | 3/9/04 |
| HorizonsDoc.cpp | 1,153 | 3/9/04 |
| HorizonsDoc.h | 627 | 3/9/04 |
| HorizonsHomeView.cpp | 2,174 | 3/9/04 |
| HorizonsHomeView.h | 794 | 3/9/04 |
| HorizonsView.cpp | 1,840 | 3/9/04 |
| HorizonsView.h | 988 | 3/9/04 |
| ImageBrowser.cpp | 17,280 | 3/9/04 |
| ImageBrowser.h | 2,128 | 3/9/04 |
| ImageLocator10Page.cpp | 1,740 | 3/9/04 |
| ImageLocator10Page.h | 615 | 3/9/04 |
| ImageLocator20Page.cpp | 2,091 | 3/9/04 |
| ImageLocator20Page.h | 638 | 3/9/04 |
| ImageLocator30Page.cpp | 1,874 | 3/9/04 |
| ImageLocator30Page.h | 614 | 3/9/04 |
| ImageLocatorSheet.cpp | 2,359 | 3/9/04 |
| ImageLocatorSheet.h | 1,057 | 3/9/04 |
| ImageSelect.cpp | 22,275 | 3/9/04 |
| ImageSelect.h | 2,239 | 3/9/04 |
| ImageSelect.htm | 358 | 3/9/04 |
| ImageViewer.cpp | 22,873 | 3/9/04 |
| ImageViewer.h | 2,458 | 3/9/04 |
| LeftView.cpp | 22,140 | 3/9/04 |
| LeftView.h | 2,047 | 3/9/04 |
| LoginDlg.cpp | 4,158 | 3/9/04 |
| LoginDlg.h | 863 | 3/9/04 |
| MainFrm.cpp | 28,529 | 3/9/04 |
| MainFrm.h | 2,615 | 3/9/04 |
| MySplitterWnd.cpp | 2,577 | 3/9/04 |
| MySplitterWnd.h | 625 | 3/9/04 |
| OverviewView.cpp | 56,439 | 3/9/04 |
| OverviewView.h | 2,379 | 3/9/04 |
| ReadMe.txt | 6,666 | 3/9/04 |
| ReportsOverviewView.cpp | 37,388 | 3/9/04 |
| ReportsOverviewView.h | 1,963 | 3/9/04 |
| resource.h | 22,889 | 3/9/04 |
| resource.h_old | 14,820 | 3/9/04 |
| SelfDesc10Page.cpp | 2,384 | 3/9/04 |
| SelfDesc10Page.h | 732 | 3/9/04 |
| SelfDesc20Page.cpp | 8,354 | 3/9/04 |
| SelfDesc20Page.h | 1,270 | 3/9/04 |
| SelfDesc25Page.cpp | 14,817 | 3/9/04 |
| SelfDesc25Page.h | 1,613 | 3/9/04 |
| SelfDesc30Page.cpp | 4,766 | 3/9/04 |
| SelfDesc30Page.h | 943 | 3/9/04 |
| SelfDesc40Page.cpp | 8,735 | 3/9/04 |
| SelfDesc40Page.h | 1,147 | 3/9/04 |
| SelfDesc45Page.cpp | 5,470 | 3/9/04 |
| SelfDesc45Page.h | 926 | 3/9/04 |
| SelfDesc50Page.cpp | 1,663 | 3/9/04 |
| SelfDesc50Page.h | 536 | 3/9/04 |
| SelfDesc60Page.cpp | 4,811 | 3/9/04 |
| SelfDesc60Page.h | 950 | 3/9/04 |
| SelfDesc70Page.cpp | 5,070 | 3/9/04 |
| SelfDesc70Page.h | 919 | 3/9/04 |
| SelfDesc80Page.cpp | 6,556 | 3/9/04 |
| SelfDesc80Page.h | 1,115 | 3/9/04 |
| SelfDesc85Page.cpp | 4,117 | 3/9/04 |
| SelfDesc85Page.h | 855 | 3/9/04 |
| SelfDesc90Page.cpp | 1,965 | 3/9/04 |
| SelfDesc90Page.h | 624 | 3/9/04 |
| SelfDescSheet.cpp | 3,230 | 3/9/04 |
| SelfDescSheet.h | 1,684 | 3/9/04 |
| ShellFolders.cpp | 4,083 | 3/9/04 |
| ShellFolders.h | 726 | 3/9/04 |
| SlideShowImageSelect.cpp | 19,737 | 3/9/04 |
| SlideShowImageSelect.h | 2,018 | 3/9/04 |
| stdafx.cpp | 208 | 3/9/04 |
| stdafx.h | 1,972 | 3/9/04 |
| TableReporterHTMLView.cpp | 15,076 | 3/9/04 |
| TableReporterHTMLView.h | 2,654 | 3/9/04 |
| TableReporterHTMLView_{—} | 7,754 | 3/9/04 |
| CompletedAffirmations.cpp | | |
| TableReporterHTMLView_{—} | 728 | 3/9/04 |
| CompletedAffirmations.h | | |
| TableReporterHTMLView_CompletedGoals.cpp | 6,553 | 3/9/04 |
| TableReporterHTMLView_CompletedGoals.h | 609 | 3/9/04 |
| TableReporterHTMLView_DailyExercises.cpp | 6,589 | 3/9/04 |
| TableReporterHTMLView_DailyExercises.h | 769 | 3/9/04 |
| TableReporterHTMLView_DaysPracticed.cpp | 6,484 | 3/9/04 |
| TableReporterHTMLView_DaysPracticed.h | 785 | 3/9/04 |
| TableReporterHTMLView_{—} | 8,390 | 3/9/04 |
| DefinedAffirmations.cpp | | |
| TableReporterHTMLView_DefinedAffirmations.h | 716 | 3/9/04 |
| TableReporterHTMLView_DefinedGoals.cpp | 7,123 | 3/9/04 |
| TableReporterHTMLView_DefinedGoals.h | 601 | 3/9/04 |
| TableReporterHTMLView_{—} | 4,227 | 3/9/04 |
| FeedbackLogByDate.cpp | | |
| TableReporterHTMLView_FeedbackLogByDate.h | 692 | 3/9/04 |
| TableReporterHTMLView_{—} | 2,423 | 3/9/04 |
| FeedbackLogByGoal.cpp | | |
| TableReporterHTMLView_FeedbackLogByGoal.h | 442 | 3/9/04 |
| TableReporterHTMLView_ProgressLogByDate.cpp | 4,227 | 3/9/04 |
| TableReporterHTMLView_ProgressLogByDate.h | 692 | 3/9/04 |
| TableReporterHTMLView_{—} | 2,423 | 3/9/04 |
| ProgressLogByGoal.cpp | | |
| TableReporterHTMLView_ProgressLogByGoal.h | 442 | 3/9/04 |
| TableReporterHTMLView_SelfDescription.cpp | 15,549 | 3/9/04 |
| TableReporterHTMLView_SelfDescription.h | 1,035 | 3/9/04 |
| TabView.cpp | 12,766 | 3/9/04 |
| TabView.h | 2,279 | 3/9/04 |
| Test.htm | 0 | 3/9/04 |
| VisualBuilder10Page.cpp | 1,756 | 3/9/04 |
| VisualBuilder10Page.h | 628 | 3/9/04 |
| VisualBuilder20Page.cpp | 2,109 | 3/9/04 |
| VisualBuilder20Page.h | 651 | 3/9/04 |
| VisualBuilder30Page.cpp | 1,887 | 3/9/04 |
| VisualBuilder30Page.h | 653 | 3/9/04 |
| VisualBuilder40Page.cpp | 1,885 | 3/9/04 |
| VisualBuilder40Page.h | 628 | 3/9/04 |
| VisualBuilder50Page.cpp | 1,838 | 3/9/04 |
| VisualBuilder50Page.h | 627 | 3/9/04 |
| VisualBuilder60Page.cpp | 1,840 | 3/9/04 |
| VisualBuilder60Page.h | 627 | 3/9/04 |
| VisualBuilderSheet.cpp | 2,707 | 3/9/04 |
| VisualBuilderSheet.h | 1,368 | 3/9/04 |
| VisualizationExercise.cpp | 12,141 | 3/9/04 |
| VisualizationExercise.h | 1,584 | 3/9/04 |
| VisualizationExercise.htm | 1,924 | 3/9/04 |
| VisualizationMaintenance.cpp | 16,340 | 3/9/04 |
| VisualizationMaintenance.h | 1,936 | 3/9/04 |
| VisualizationMaintenance.htm | 1,923 | 3/9/04 |
| WindowFont.h | 2,665 | 3/9/04 |
| _DlgEnterAffirmations.cpp_od | 25,420 | 3/9/04 |
| _DlgenterAffirmations.htm_old | 2,022 | 3/9/04 |
| _DlgEnterAffirmations.h_old | 2,570 | 3/9/04 |
| afx_hidd_color.htm | 380 | 3/9/04 |
| afx_hidd_fileopen.htm | 1,123 | 3/9/04 |
| afx_hidd_filesave.htm | 1,172 | 3/9/04 |
| afx_hidd_find.htm | 363 | 3/9/04 |
| afx_hidd_font.htm | 377 | 3/9/04 |
| afx_hidd_newtypedlg.htm | 554 | 3/9/04 |
| afx_hidd_replace.htm | 372 | 3/9/04 |
| afx_hidp_default.htm | 691 | 3/9/04 |
| afx_hidw_dockbar_top.htm | 556 | 3/9/04 |
| afx_hidw_status_bar.htm | 1,558 | 3/9/04 |
| afx_hidw_toolbar.htm | 744 | 3/9/04 |
| hidr_doc1type.htm | 1,277 | 3/9/04 |
| hid_app_about.htm | 440 | 3/9/04 |
| hid_app_exit.htm | 699 | 3/9/04 |
| hid_context_help.htm | 684 | 3/9/04 |
| hid_edit_clear.htm | 381 | 3/9/04 |
| hid_edit_clear_all.htm | 385 | 3/9/04 |
| hid_edit_copy.htm | 549 | 3/9/04 |
| hid_edit_cut.htm | 589 | 3/9/04 |
| hid_edit_find.htm | 378 | 3/9/04 |
| hid_edit_paste.htm | 472 | 3/9/04 |
| hid_edit_redo.htm | 385 | 3/9/04 |
| hid_edit_repeat.htm | 497 | 3/9/04 |
| hid_edit_replace.htm | 387 | 3/9/04 |
| hid_edit_undo.htm | 751 | 3/9/04 |
| hid_file_close.htm | 936 | 3/9/04 |
| hid_file_mru_file1.htm | 692 | 3/9/04 |
| hid_file_new.htm | 864 | 3/9/04 |
| hid_file_open.htm | 824 | 3/9/04 |
| hid_file_save.htm | 890 | 3/9/04 |
| hid_file_save_as.htm | 795 | 3/9/04 |
| hid_file_send_mail.htm | 677 | 3/9/04 |
| hid_help_index.htm | 661 | 3/9/04 |
| hid_help_using.htm | 394 | 3/9/04 |
| hid_ht_caption.htm | 1,209 | 3/9/04 |
| hid_ht_nowhere.htm | 366 | 3/9/04 |
| hid_next_pane.htm | 363 | 3/9/04 |
| hid_prev_pane.htm | 363 | 3/9/04 |
| hid_sc_close.htm | 707 | 3/9/04 |
| hid_sc_maximize.htm | 421 | 3/9/04 |
| hid_sc_minimize.htm | 423 | 3/9/04 |
| hid_sc_move.htm | 525 | 3/9/04 |
| hid_sc_nextwindow.htm | 548 | 3/9/04 |
| hid_sc_prevwindow.htm | 561 | 3/9/04 |
| hid_sc_restore.htm | 481 | 3/9/04 |
| hid_sc_size.htm | 830 | 3/9/04 |
| hid_sc_tasklist.htm | 1,446 | 3/9/04 |
| hid_view_ruler.htm | 381 | 3/9/04 |
| hid_view_status_bar.htm | 848 | 3/9/04 |
| hid_view_toolbar.htm | 814 | 3/9/04 |
| hid_window_all.htm | 599 | 3/9/04 |
| hid_window_arrange.htm | 613 | 3/9/04 |
| hid_window_cascade.htm | 423 | 3/9/04 |
| hid_window_new.htm | 748 | 3/9/04 |
| hid_window_split.htm | 766 | 3/9/04 |
| hid_window_tile.htm | 415 | 3/9/04 |
| hid_window_tile_horz.htm | 455 | 3/9/04 |
| hid_window_tile_vert.htm | 424 | 3/9/04 |
| Horizons.chm | 29,262 | 3/9/04 |
| Horizons.hhc | 1,350 | 3/9/04 |
| Horizons.hhk | 203 | 3/9/04 |
| Horizons.hhp | 6,763 | 3/9/04 |
| HTMLDefines.h | 22,543 | 3/9/04 |
| main_index.htm | 672 | 3/9/04 |
| menu_edit.htm | 1,043 | 3/9/04 |
| menu_file.htm | 1,448 | 3/9/04 |
| menu_help.htm | 799 | 3/9/04 |
| menu_view.htm | 704 | 3/9/04 |
| menu_window.htm | 1,291 | 3/9/04 |
| scrollbars.htm | 672 | 3/9/04 |
| corner_left.gif | 69 | 3/9/04 |
| corner_right.gif | 70 | 3/9/04 |
| header_bkgnd.gif | 170 | 3/9/04 |
| header_overview.gif | 8,074 | 3/9/04 |
| overview_logo.gif | 24,486 | 3/9/04 |
| spacer.gif | 1,082 | 3/9/04 |
| star_contemplation.gif | 1,478 | 3/9/04 |
| Affirmation.ico | 28,902 | 3/9/04 |
| Checkmrk.ico | 1,078 | 3/9/04 |
| Closed folder.ico | 24,190 | 3/9/04 |
| Completed Affirmation.ico | 21,462 | 3/9/04 |
| Completed Goals Folder.ico | 24,190 | 3/9/04 |
| Contemplation.ico | 28,902 | 3/9/04 |
| Copy of DSNBuddy.ico | 2,238 | 3/9/04 |
| FONTO1.ico | 1,846 | 3/9/04 |
| Goal 2.ico | 24,190 | 3/9/04 |
| Goal.ico | 28,902 | 3/9/04 |
| Help.ico | 17,694 | 3/9/04 |
| icon1.ico | 2,238 | 3/9/04 |
| icon2.ico | 2,238 | 3/9/04 |
| INFO.ico | 1,846 | 3/9/04 |
| JavaCup.ico | 22,206 | 3/9/04 |
| MISC02.ico | 1,078 | 3/9/04 |
| MyFirstIcon.ico | 22,486 | 3/9/04 |
| New Goal.ico | 894 | 3/9/04 |
| NewGoal.bmp | 1,334 | 3/9/04 |
| Open Completed Goals Folder.ico | 21,462 | 3/9/04 |
| Open Folder.ico | 24,190 | 3/9/04 |
| Open Report Folder.ico | 21,462 | 3/9/04 |
| Report Folder.ico | 21,462 | 3/9/04 |
| Report.ico | 24,190 | 3/9/04 |
| single-step.ico | 4,662 | 3/9/04 |
| ss icon dark.ico | 4,286 | 3/9/04 |
| ss icon.ico | 22,486 | 3/9/04 |
| Success.ico | 17,694 | 3/9/04 |
| thoughts.ico | 21,462 | 3/9/04 |
| Visualization.ico | 28,902 | 3/9/04 |
| W95MBX01.ICO | 1,078 | 3/9/04 |
| bigvf.bmp | 91,198 | 3/9/04 |
| bigvf.gif | 2,172 | 3/9/04 |
| bigvf2.bmp | 25,846 | 3/9/04 |
| bitmap1.bmp | 246 | 3/9/04 |
| cathedral.jpg | 22,497 | 3/9/04 |
| Copy of side-bar---curve.bmp | 87,478 | 3/9/04 |
| goal.bmp. | 131,702 | 3/9/04 |
| goal.ico | 3,262 | 3/9/04 |
| goal.jpg | 11,406 | 3/9/04 |
| goal2.bmp | 131,702 | 3/9/04 |
| goal3.bmp | 129,862 | 3/9/04 |
| graph.bmp | 397,878 | 3/9/04 |
| graph.gif | 14,722 | 3/9/04 |
| graph2.bmp | 50,358 | 3/9/04 |
| header_overview.gif | 6,754 | 3/9/04 |
| horizons.gif | 2,102 | 3/9/04 |
| horizons.ico | 2,238 | 3/9/04 |
| horizons.manifest | 698 | 3/9/04 |
| horizons.rc2 | 399 | 3/9/04 |
| horizons1.gif | 980 | 3/9/04 |
| horizons2.gif | 2,102 | 3/9/04 |
| horizonsDoc.ico | 1,078 | 3/9/04 |
| horizonsHomePage.htm | 1,656 | 3/9/04 |
| horizons_old.ico | 766 | 3/9/04 |
| horizons_.ico | 4,662 | 3/9/04 |
| html1.htm | 0 | 3/9/04 |
| html_ima.htm | 1,915 | 3/9/04 |
| ImageSrcs.txt | 225 | 3/9/04 |
| ImageViewer.htm | 1,915 | 3/9/04 |
| log_color_bmp.gif | 12,562 | 3/9/04 |
| motivation.bmp | 138,774 | 3/9/04 |
| motivation.jpg | 12,993 | 3/9/04 |
| newwishingtowers.bmp | 564,054 | 3/9/04 |
| newwishingtowers1.bmp | 157,994 | 3/9/04 |
| newwishingtowers10b.jpg | 28,731 | 3/9/04 |
| newwishingtowers2.bmp | 139,974 | 3/9/04 |
| overview.htm | 2,321 | 3/9/04 |
| overview_logo.gif | 45,974 | 3/9/04 |
| ReportsOverview.htm | 7,044 | 3/9/04 |
| side-bar---curve.bmp | 16,258 | 3/9/04 |
| side-bar---curve.gif | 8,518 | 3/9/04 |
| side-bar---curve2.bmp | 39,670 | 3/9/04 |
| side-bar-curve2a.bmp | 43,638 | 3/9/04 |
| single-step overview small.gif | 31,678 | 3/9/04 |
| single-step reports small.gif | 31,678 | 3/9/04 |
| single-step.ico | 4,662 | 3/9/04 |
| Toolbar.bmp | 1,678 | 3/9/04 |
| Vfdraw.jpg | 758 | 3/9/04 |
| Bullet.gif | 816 | 3/9/04 |

### BACKGROUND OF THE INVENTION

1. Field of the Invention

The present invention in its various aspects generally relates to the fields of psychology, education, personal health care, and emotional and motivational therapy, as well as to data collection and retrieval systems and methods.

2. Description of the Related Art

Historically, it was believed that rational thought played the greatest role in an individual's success and happiness in life. Psychologists are now coming to the realization that emotions also play a large if not larger role in an individual's successful, happy life. Emotions tend to be viewed as natural consequences to events we experience every day in our lives. In fact, we have the capacity and capability to control our emotions. One's ability or lack thereof to control our emotions is typically learned at a young age from parents and friends. These early “learned” emotional control lessons often follow us through life unless positive steps are taken to change them.

“Negative” emotions tend to hinder or detract from rational thought. An example of a negative emotion might be the fear of an elevator experienced by someone who has learned to fear elevators from a past negative experience in one. An individual may act in a manner that is not in his best interests due to an uncontrolled emotional response. An individual whose emotions cause him to react to certain situations in a negative manner may desire to change that learned reaction through control of his emotions.

Emotions can be helpful or therapeutic when summoned at the appropriate time. These emotions may be positive, such as reminding oneself of a loved one far away or of one's children. These emotions may also be negative, as discussed above, for example when a therapist must bring an individual back to a painful memory to help the individual work through the feelings. It would therefore be useful to have the abilities to control emotions and evoke desired emotions at a specific time.

Most people are quite skilled at managing some of their emotions, but this management tends to take place below a conscious level. We may eat chocolate, or go shopping when we're feeling down, drive too fast when we're feeling angry, take a walk when we're feeling frustrated, etc. It would be beneficial to be able control a wide range of emotions in a more controlled manner.

An emotional response is often initiated by external stimuli to which a person is subjected. Accordingly, media containing external stimuli have been used in various environments to control emotions of a given audience. However, emotional responses to a given stimuli vary from individual to individual. A given media presentation may be interpreted negatively by one individual, neutrally by a second individual, and positively by a third individual. For example, a picture of a mountain may evoke a positive feeling of exhilaration in a mountain climber, but a negative feeling of fear in a person having acrophobia. These different and in some cases divergent interpretations may vary based on one's personal experiences, personality, temperament, and learned emotional control abilities. The intensity of an emotional response also varies from individual to individual, and may manifest itself differently from individual to individual, e.g., from a tightening of various muscles, a change in tension of the skin, a secretion of hormones, an increased pulse rate, a change in brain activity, etc.

3. Objects of the Invention

It is an object of the invention to provide a method and/or system for compiling or organizing electronically recallable media samples into one or more corresponding emotional state categories based on the personal emotional responses an individual provides to the media samples.

It is a further object of the invention to provide a method and/or system for evoking an emotional state in an individual through presentation of electronically recallable media samples selectively organized by the individual into one or more corresponding emotional state categories.

It is another object of this invention to provide a method and/or system for assisting an individual to reach a target goal by presenting the individual with an electronically recallable media sample during performance and/or upon completion of an exercise to evoke and emotional state.

### SUMMARY OF THE INVENTION

To achieve one or more of the foregoing objects, and in accordance with the purposes of the invention as embodied and broadly described in this document, according to a first aspect of this invention there is provided a method of categorizing electronically recallable media, comprising presenting a particular user with media samples possessing subject matter containing sensory stimuli. The particular user is permitted to provide personal responses to the sensory stimuli of the media samples. From the personal responses, a determination is made whether the media samples evoke a selected emotional state in the particular user. The media samples evoking the selected emotional state in the particular user are assigned to an electronically recallable set, the electronically recallable set being labeled with an emotional state identifier representative of the selected emotional state.

A second aspect of the invention provides a method of categorizing electronically recallable media. According to the method, a particular user is presented with media samples possessing subject matter containing sensory stimuli, and permitted to provide personal responses to the sensory stimuli of the media samples. From the personal responses, it is determined whether the media samples evoke one or more selected emotional states in the particular user. Media samples are electronically assigned to electronically recallable sets corresponding to the emotional states that the media samples evoked in the particular user, each of the electronically recallable sets being labeled with a respective emotional state identifier representative of at least one of the selected emotional states.

According to a third aspect of the invention, a method is provided for evoking an emotional state in a particular user through presentation of media samples. The method comprises providing an electronically recallable set of self-selected media samples custom selected for evoking the emotional state in the particular user, and, at a selected time to evoke the emotional state, electronically presenting the particular user with the media sample.

A fourth aspect of the invention provides a computer usable medium for compiling electronically recallable sets of media samples customized for evoking corresponding emotional states in a particular user. The computer usable medium comprises:
(a) computer readable program code means for causing a computer to display a category entry field to a particular user via a display unit;
(b) computer readable program code means for causing the computer to receive an inputted electronically recallable category or categories into the category entry field, said electronically recallable category or categories each corresponding to a respective emotional state;
(c) computer readable program code means for presenting the particular user with a plurality of media samples, the media samples possessing subject matter containing sensory stimuli;
(d) computer readable program code means for permitting the media samples to be electronically assigned to the electronically recallable category or one or more of the electronically recallable categories based on the emotional states evoked in the particular user by the media samples; and
(e) computer readable program code means for permitting the particular user to electronically recall the media samples by the electronically recallable category or categories.

A fifth aspect of the invention provides a system for compiling electronically recallable sets of media customized for evoking corresponding emotional states in a particular user. The system comprises a computing device comprising the computer program, an input device, an output device, and data storage. The computer program provides a particular user with a plurality of categories each corresponding to a respective emotional state, obtains from the data storage media samples possessing subject matter containing sensory stimuli, presents the media samples to the particular user via the output device and permits the particular user to provide personal responses to the media samples, electronically assigns the media samples via the input device into one or more electronically recallable sets of the categories based on emotional states evoked by the media samples, and permits selective electronic recall of the media samples.

### BRIEF DESCRIPTION OF THE DRAWINGS

The accompanying drawings are incorporated in and constitute a part of the specification. The drawings, together with the general description given above and the detailed description of the preferred embodiments and methods given below, serve to explain the principles of the invention. In such drawings:

FIG. 1 is a flow chart of an embodiment of the invention illustrating a method for compiling electronically recallable sets of media customized for evoking corresponding emotional states in a particular user;

FIG. 2 is a block diagram representing a computer system of an embodiment of the present invention;

FIGS. 3 through 9 are user interface screen views of a computer program embodying a method of the present invention;

FIGS. 10 through 22 are user interface screen views of another computer program embodying a method of the present invention.

### DETAILED DESCRIPTION OF CERTAIN PREFERRED EMBODIMENTS AND METHODS OF THE INVENTION

Reference will now be made in detail to the presently preferred embodiments and methods of the invention as illustrated in the accompanying drawings, in which like reference characters designate like or corresponding parts throughout the drawings. It should be noted, however, that the invention in its broader aspects is not limited to the specific details, representative devices and methods, and illustrative examples shown and described in this section in connection with the preferred embodiments and methods. The invention according to its various aspects is particularly pointed out and distinctly claimed in the attached claims read in view of this specification, and appropriate equivalents.

It is to be noted that, as used in the specification and the appended claims, the singular forms “a,” “an,” and “the” include plural referents unless the context clearly dictates otherwise.

As referred to herein, the term “emotional state” comprises a mental state arising subjectively (and not necessarily through conscious effort) and is accompanied by physiological changes.

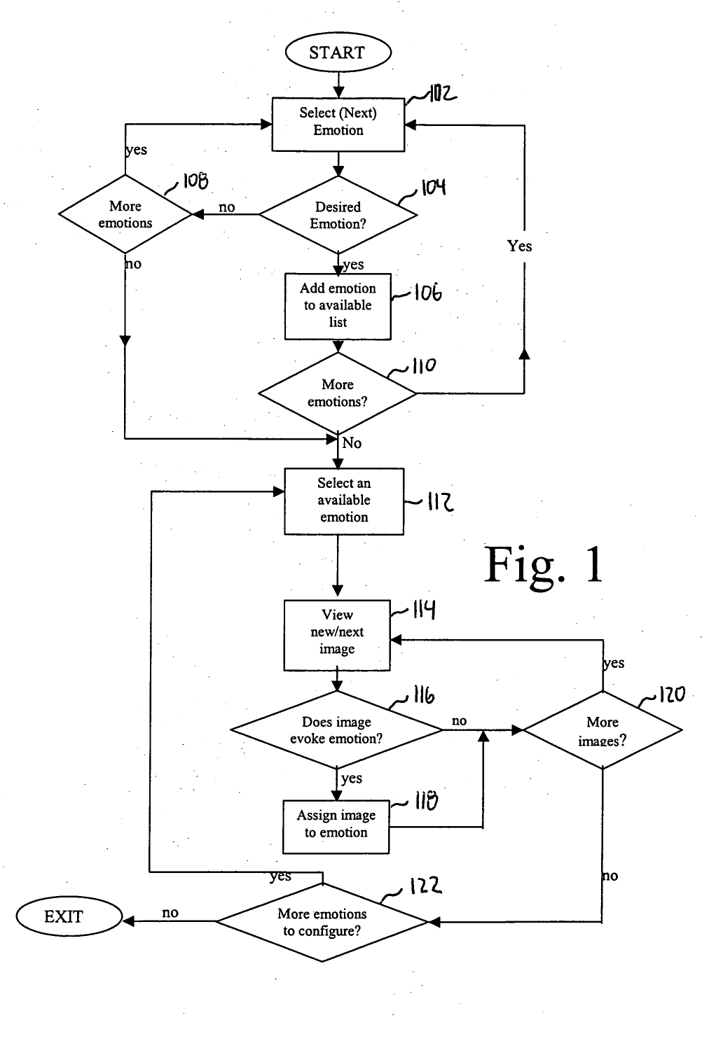

FIG. 1 illustrates a flow diagram of an embodied method for compiling an electronically recallable set of media samples customized for evoking a selected emotional state in a particular user. According to the illustrated embodiment, the method comprises categorizing media samples into electronically recallable sets, each of the electronically recallable sets corresponding to a respective emotional state.

Referring more particularly to FIG. 1, the desired emotional states are first selected. In the illustrated embodiment, the selection step 102 comprises accessing a list of “available” emotional (or mental) states, which are preferably pre-programmed into a computer program. Selectable emotional states might include, for example and not necessarily by limitation, positive states (e.g., motivated, excited, joyful, nostalgic, peaceful, happy, patriotic) and negative states (e.g., pressured, fearful, anxious, threatened, angered, frustrated, hungry, sad). This list is exemplary, and not exhaustive of possible emotions that may be selected. Other emotional states include surprise, disgust, acceptance, and anticipation. Each of the emotional states in the list may be labeled or otherwise designated with an “emotional state identifier” comprising a word, name, description, definition, symbol, image (e.g., facial image, such as a smiley face for a happy emotional state), etc. generally or universally representative of the emotional state. Alternatively, the “identifier” may represent something other than the emotional state, so long as the identifier has a conscious or unconscious meaning to the particular user associated with an emotional state.

Selection may comprise selection of one or more positive emotional states and/or one or more negative emotional states. The available emotional states may be presented collectively, e.g., as members of an inventory or list, such as a drop-down list. As an alternative, the available emotional states may be presented one-by-one on an individual basis or in sets, and accepted or rejected individually or in sets by the selecting person. The person deciding whether to accept or reject an available emotional state (i.e., “the selector”) may be the same person for whom the program is designed, i.e., the particular user whose personal responses dictate the assignment of media samples to corresponding emotional states. Alternatively, the selector may be a third person, such as a counselor, teacher, or parent.

In FIG. 1, for each available emotional state, a decision 104 is made whether to accept or reject the available emotional state for a particular application or user. This decision process 104 may involve an affirmative step on the part of the selector, e.g., in entering a command via keystroke or mouse click to accept or reject a given choice. It is also within the scope of this invention for the selection process to comprise removing rejected or unwanted emotional states from the list, so that accepted emotional states remain.

If the selector chooses to accept a given available emotional state, then the available emotional state is placed into a “selected” list 106. The selector is faced with a decision 108 and 110 subsequent to steps 104 and 106, respectively, as to whether to select additional emotional states from the available list. This selection process may be repeated once, twice, or a greater plurality of times until the decision 108 is made not to select further emotional states. The decision to cease selection of emotional states may be made after one, a plurality, or all of the desired selections from the available emotional state choices have been considered for selection. It is within the scope of this invention for the selection process to comprise selection of a single available emotional state. In this regard, separate programs may be provided for evoking different emotional states.

Although not illustrated in the embodiment of FIG. 1, the computer program user may optionally input emotional states not pre-programmed into the computer program. That is, the computer program may use a combination of pre-programmed emotional states and user-programmed emotional states. As a further embodiment, the computer program may be written to receive emotional states solely from the particular user, e.g., with no preexisting drop-down list. Another embodiment comprises operating assigning media samples to a single emotional state (instead of a plurality of emotional states), in which case the step of selecting emotional states may be omitted.

The next step comprises making the media samples available to the particular user. The media samples possess subject matter containing sensory stimuli capable of evoking an emotional state in the particular user. As referred to herein, “media” refers to a means of communication through which subject matter containing the sensory stimuli is presented to the particular user. The media samples may be in the form of a visual, audio, olfactory, or sensory sample, or a combination thereof (e.g., a video-audio), as well as other known media. For example, visual media samples may take the form of a photograph, drawing, picture, painting, video, image, character, symbol, or electronic presentation. A visual media sample may include text (e.g., a word, phrase, or sentence), a text-free image, or a combination thereof, e.g., text superimposed over an image. In the case of a textual media sample, the text may comprise an affirmation. An audio media sample may take the form of a jingle, song or song excerpt, spoken affirming word or statement, a sound bite of a natural occurrence (e.g., wind, water, waves) or artificial noise (e.g., car engine, fog horn), or the like. Olfactory media samples may comprise a familiar or pleasant smell. Other sensory media samples include tactile and gustatory senses.

A given media sample itself may or may not constitute an electronic file. However, the media samples are electronically recallable to permit the media samples to be summoned/retrieved by electronic signal. For example, to provide an olfactory media, a series of perfume samples could be suspended in cotton in individually sealed containers. The containers could have portals openable via relays when signaled, e.g., by a computer. A selection of samples could be provided in sequence to a user who might select which samples evoked emotional memories (the perfume worn by the user's mother perhaps). A similar setup might provide edible samples directly to the mouth of a user alone or in combination with another signal, such as a wireless or pneumatic signal.

As evident from the above examples, electronic recall of media samples may further comprise use of a non-electronic signal in conjunction with the electronic signal. For example, electronic recall may comprise using a pneumatic signal (e.g., air jets), vibrotactile stimulation, electrotactile stimulation, functional neuromuscular stimulation. The user may generate the signal using various devices, such as via a keyboard, mouse, force-reflecting joystick, haptic feedback devices, robotics and exoskeletons, etc.

The source of media samples is generally not restricted. Media samples may be obtained from a single source or a plurality of different sources. A given source may contain only one available media sample or a plurality of media samples. The source may comprise a random or organized collection of media samples. The source may be publicly accessible, such as over the internet, or may comprise a collection recorded or otherwise stored on appropriate medium, such as a digital disk, hard disk, tape, or diskette. The media sample source may comprise items belonging, obtained from, or created by or for the particular user. Media samples may be placed into an electronically recallable format through the use of, for example, audio recorders, photography equipment (e.g., digital cameras), videography equipment, scanners, and the like.

The particular user organizes the media samples into corresponding sets or categories based on the emotional responses evoked the media sample evoke in the particular user. Returning to FIG. 1, the particular user performs a step 112 of selecting an emotional category for configuration. The media samples are presented 114 to the particular user. Presentation may be performed individually, i.e., one media sample at a time in a random or ordered sequence. Alternatively, the particular user may be presented with a plurality or all of the media samples to select from at once.

The particular user is afforded an opportunity to provide a response relating to whether the media samples evoke the selected emotion. The particular user is charged with this task because the personal responses evoked by the media samples are largely subjective in nature. For example, a given media sample may evoke a positive response or mental state in one individual, but a negative response or metal state in another individual. The personal response may comprise, for example, an emotional, psychological, physical, or physiological response of the particular user. The personal response may manifest itself as an outward expression or action observable to an evaluator, or as an inward feeling or sensation discernible only to the particular user.

Based on the personal response, a determination 116 is made as to whether the media sample evokes one or more (or none) of the selected emotional states in the particular user. The particular user may be given sole or partial responsibility in making this determination based on a set of predefined criteria or on the subjective opinion of the particular user. Alternatively, responsibility in making this determination may be shared with or placed exclusively in the jurisdiction of another person, such as a counselor or evaluator, who may observe the personal response and, based on that observation, make a determination of the emotional state generated by the media sample.

Another embodiment for making this determination 116 comprises defining a criterion or set of criteria, measuring the personal response, and comparing the measurement against the predefined criteria. According to this embodiment, the personal response preferably comprises a physiological and/or physical response of the particular user. An external device, such as a bio-feedback instrument, may be used for taking the measurement of the physiological and/or physical response. Exemplary external devices compatible with embodiments of the invention include electroencephalograph (EEG) electrodes, a positron emission tomography (PET), computerized axial tomography (CAT) scan, magnetic resonance imaging (MRI), and/or Galvanic Skin Resistance (GSR).

If the media sample evokes a personal response corresponding to the selected emotional state, then the media sample is assigned 118 to the category/set corresponding to the emotional state. On the other hand, if the media sample does not evoke a personal response corresponding to the selected emotional state, then the media sample is not assigned to the corresponding category. In either case, subsequent to steps 116 or 118 a decision 120 is made whether to present additional media samples to the particular user. If additional media samples are to be presented for possible assignment to the selected emotion, then the next media sample is viewed 114. If no additional media samples are to be presented to the particular user for a given emotion, then a decision 122 is made whether to configure other emotions by returning to step 112 and assigning media samples to a new selected emotion. These steps are repeated, preferably a sufficient number of times to assign two, three, or more media samples to each of the selected emotional states. Optionally, for each of the selected emotional states, the associated media samples may be combined into a scripted presentation or otherwise compiled.

According to a preferred embodiment of the present invention, the method further comprises updating (or reconfiguring) prior assignments of media samples. The emotional significance that a given media sample may have on a particular user may be eroded by repeated exposure, or changed, for example, due to an outside personal experience of the particular user that redefines the emotional significance of the subject matter of the media sample in the mind of the particular user. Further, recent events in the user's life may create an emotional response to a previously insignificant media sample.

The updating (or reconfiguration) step may comprise, for example, reviewing media samples in an emotional state category to re-determine whether the media samples still evoke the corresponding emotional state in the particular user, and removing media samples from the emotional state category that no longer evoke the corresponding emotional state in the particular individual. Additionally or alternatively, the updating step may comprise, for example, supplementing an existing emotional state category with new media sample or creating a new emotional state category and filling it with media samples. The updating step may be repeated on a random or periodic schedule, e.g., monthly, semi-annually, or annually.

Yet another related embodiment of the present invention, a method is provided for enabling a user to control, e.g., change, his/her emotional state by using a personally customized, electronically recallable set of self-selected media samples to evoke a desired emotion at a specific time.

The electronically recallable set of self-selected media samples used in this embodiment may be generated and maintained as described above. The media sample set may then be used in a variety of situations and applications to summon a given emotional state in the individual. In an embodiment of the invention, the particular user views, senses, or otherwise experiences one or more media samples in a given emotional state category to enable the user to summon the corresponding emotional state. The media samples may be presented individually until the corresponding emotional state is attained, or may be combined or scripted together for concurrent or successive presentation until the emotional state is attained. Different types of media samples—e.g., sound and visual—may be presented concurrently, i.e., superimposed over one another.

An example of such a situation is an athlete attempting to reach an energized or stimulated state before participating in a competition. The athlete may select a category corresponding to an emotion that will improve performance, and review electronically recallable media samples associated with category to improve performance. It is to be understood that for this and other situations/applications in which this method is used, the selection of a preferred or optimal emotional state for a given situation is dependent upon the particular user involved. What might assist one athlete in competition performance might stifle another athlete. For example, a sensation of fear may motivate one athlete into performing optimally to avoid failure, whereas fear may debilitate another athlete.

Another example of a situation in which the method of this embodiment may find use is in desensitizing an individual suffering from a phobia or other fear. The individual, i.e., particular user, may use the method of this invention to reach a desired emotional state prior to, contemporaneously with, or subsequent to encountering a fearful situation. According to an embodiment of the invention, the particular user suffering from the phobia may select a category corresponding to an emotion that will allow the individual to confront a fear, e.g., media enforcing a calming effect, and review electronically recallable media samples associated with category prior to or while encountering the fearful situation.

Still another example of a situation in which the method of this embodiment is useful is where the particular user wishes to reach a target goal. The media samples may be used to promote positive feelings in the particular user at appropriate times to promote attainment of the target goal. For example, the user or computer program may include exercises presenting the particular user with an opportunity to provide an affirmation concerning successful completion of the target goal. While performing the exercises, the media samples are electronically recalled and presented to the particular user. The target goal may comprise successful completion of a task, a change in behavior, or the cessation of an addictive behavior, such as smoking, drinking, gambling, eating, and the like. For example, in methods comprising the cessation of addictive behavior, media samples creating positive mental or emotional media samples may be presented to the user in response to the successful completion of a task, whereas negative metal or emotional media samples may be presented when the particular user considers an activity incongruous with the cessation of the additive behavior.

The electronically recallable set of media samples may also be useful in physical or mental treatment of the particular individual, such as in the case of psychological therapy. For example, a counselor or psychologist may use the electronically recallable set of media samples to summon deeply suppressed memories in the particular user. Similarly, the counselor may use the electronically recallable set of media samples to assist the particular user to reach a certain emotional state that might be helpful in his/her treatment.

The electronically recallable set(s) of media samples may also be used as a motivational tool or for spiritual inspiration.

Embodiments of systems of the present invention will now be described in detail. It is to be understood, however, that the above-described methods are not necessarily limited to practice on the systems described herein.

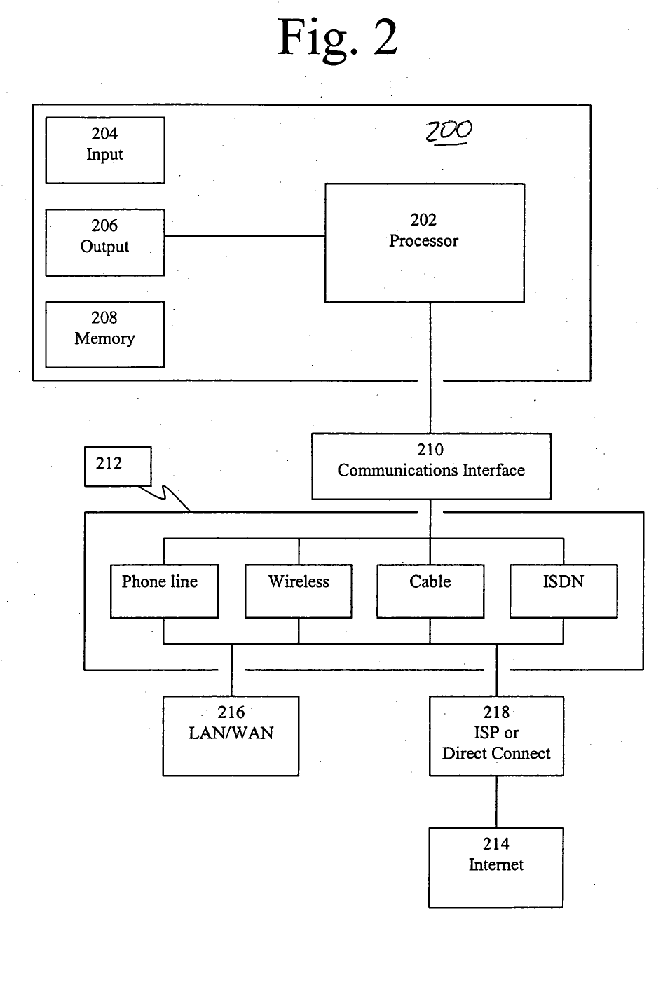

FIG. 2 is a schematic diagram of a representative embodiment of a computer system of an embodiment of the present invention. The computer system comprises a user interface terminal 200, which comprises a processor 202, such as a personal computer with a central processing unit (CPU) processor. The CPU processor may be, for example, a PENTIUM version processor from INTEL, e.g., a PENTIUM 3 or PENTIUM 4 processor running at speeds of, for example, 1.0 to 3.2 GHz, or a CELERON processor, although less or more powerful systems may be used. Other user interface terminals 200 that may be used include held-held devices, Web pads, smart phones, interactive television, interactive game consoles, two-way pagers, e-mail devices, equivalents, etc.

The processor 202 is connected electronically to an input device 204 and an output device 206. The input and output devices 204 and 206 may be integrated into or separate from the processor 202. The input device 204 may be, for example, a keyboard, a numeric or alphanumeric keypad, a pointing device (e.g., a mouse), a touch-sensitive pad, a joystick, a voice recognition system, a combination thereof, and/or other equivalent or known devices. The input device 204 generates signals in response to physical, oral, or other manipulation by the user and transmits those signals to the processor 202. The output device 206 is capable of presenting a media sample to the user. The output device 206 preferably comprises a display screen, such as a commercially available monitor, light-emitting diode (LED) display, or liquid crystal display (LCD). The output device 206 additionally or alternatively comprises any other mechanism or method for communicating with the user, such as, for example, an olfactory, visual (e.g., printer), audio (e.g., speakers), audio-visual, or other sensory device. Depending upon the intended configuration of the terminal 200, including the selected input 204 and output devices 206, a sound card (not shown) and/or a video card (not shown) may be included in the terminal 200.

The processor 202 is connected electronically to a memory 208. The memory 208 may comprise any type of computer memory and may include, for example and not necessarily by limitation, random access memory (RAM) (e.g., 256 MB of RDRAM), read-only memory (ROM), and storage device(s) such as hard disc drives and storage media, for example, optical discs and magnetic tapes and discs.

The computer program and media samples are storable in the memory 208. The media samples may be loaded into the memory 208 by various means and sources. For example, the media samples may be preloaded in the program, and/or may be stored into the memory 208 by the user by employing, for example, a digital camera, scanner, or microphone. Media samples may also be downloaded from external sources, such as through the internet.

In order to provide the terminal 200 with expanded capabilities, e.g., for internet compatibility, the terminal 200 is preferably yet optionally provided with a communications interface 210, such as network access circuitry or Ethernet network access circuitry. The communications interface 210 includes or communicates with a transmission network 212, which may include any method or system for transmitting data, including but not limited to a telephone-line modem (e.g., 56 K baud), a cable modem, a wireless connection, integrated services digital network (ISDN), or a satellite modem for connecting to the Internet 214 or a local area network (LAN), wide area network (WAN) or medium area network (MAN) 216. Wireless connections include microwave, radio frequency, and laser technologies. Connections to the Internet may be made directly or through Internet Service Providers (ISPs) 218.

The communications interface 210 and the transmission network 212 permit the user interface terminal 200 to communicate with remote data source(s) and/or server(s) 212, including databases, for example, for storing, retrieving, processing, and passing data, including media samples. The communications interface also provides the option of using the terminal 200 of the present invention as a standalone terminal or as a “dumb” terminal where the CPU processing is done remotely, e.g., at a server.

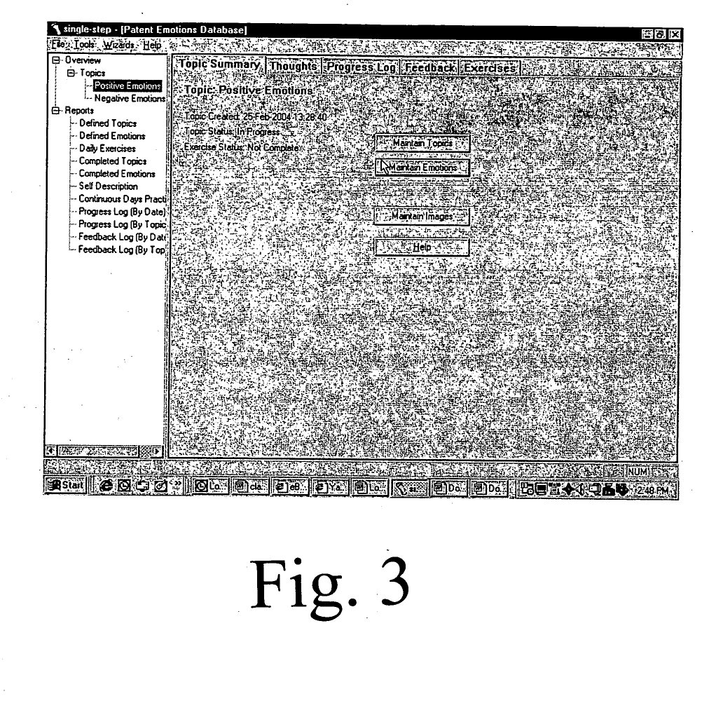

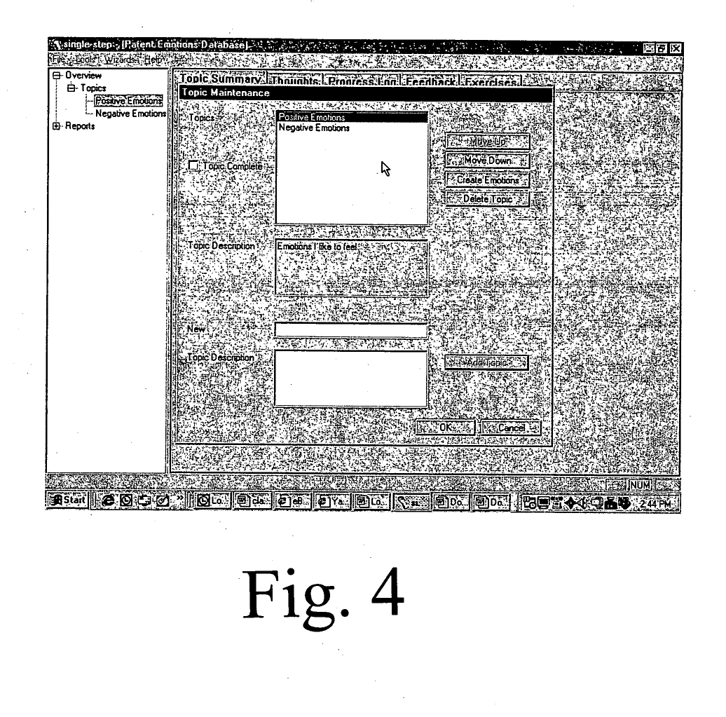

Turning now to FIGS. 3 through 9, user-interface screen printouts of a computer program capable of carrying out embodied methods of this invention are shown. Selection of the “Topics” branch from the directory tree set forth on the left side of FIG. 3 produces a listing of emotional state groupings or “topics.” The topics may be pre-entered by the software provider or may be entered by the user. For the purposes of this description, the topics “Positive Emotions” and “Negative Emotions” have been entered into the software. From the “Topic Summary” folder, the selected topics may be modified by selection (e.g., left clicking with a mouse) of the “Maintain Topics” button, which brings up the screen shown in FIG. 4. From the screen shown in FIG. 4, the topics may be manipulated. For example, the sequence or order in which the topics are listed may be changed using the “Move Up” and “Move Down” buttons. The “Delete Topic” and “Add Topic” buttons provide the user with the ability to subtract and add topics to the list.

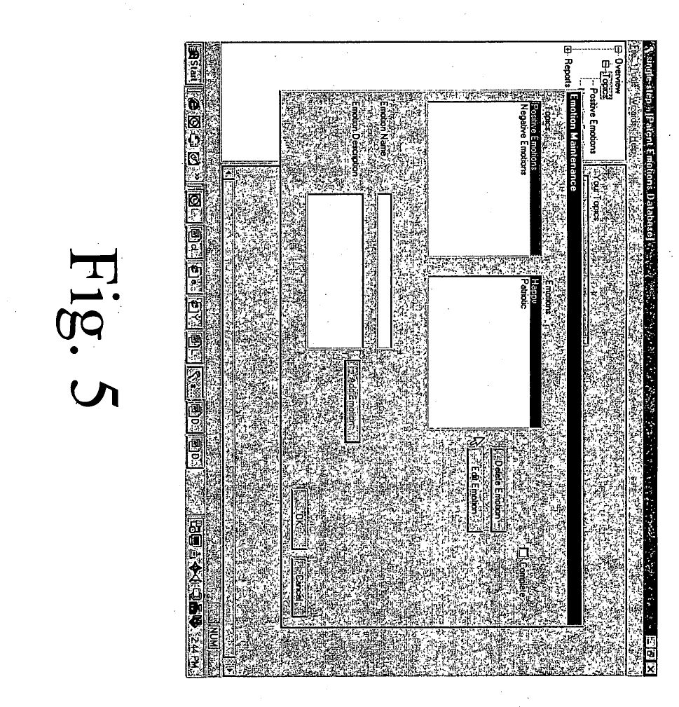

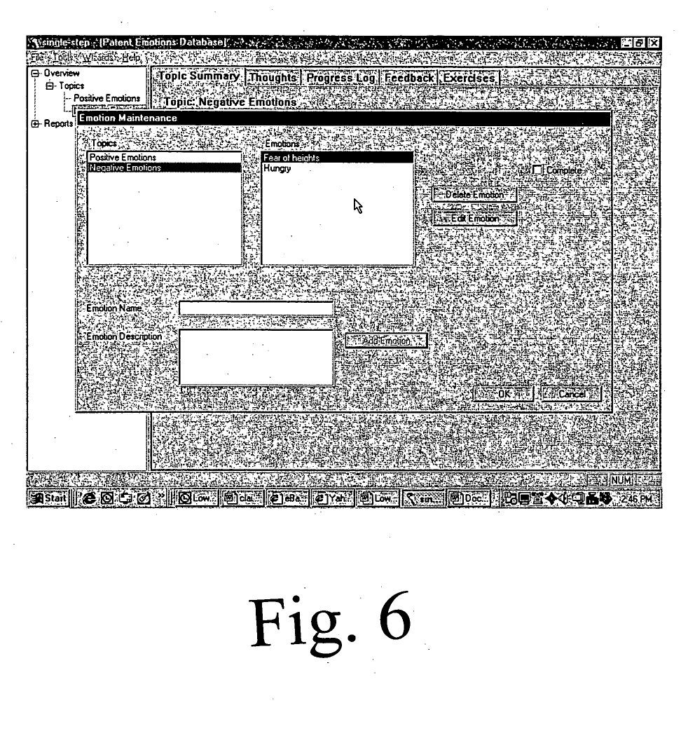

Assignment of emotional categories under these topics may be performed by selection of the “Maintain Emotions” button in FIG. 3 or the “Create Emotions” button of FIG. 4. Depending upon whether the topic “Positive Emotions” or “Negative Emotions” is highlighted, the resulting screen is shown in FIGS. 5 and 6, respectively. For the purposes of explaining the computer program, the emotions “Happy” and “Patriotic” have been pre-assigned to the Positive Emotions topic (FIG. 5), and the emotions “Fear of Heights” and “Hungry” have been pre-assigned to the Negative Emotions topic (FIG. 6). The screens shown in FIG. 5 and 6 provide the selector with the capability to delete or edit the pre-assigned emotional categories, and to add additional emotional categories under either of the topics.

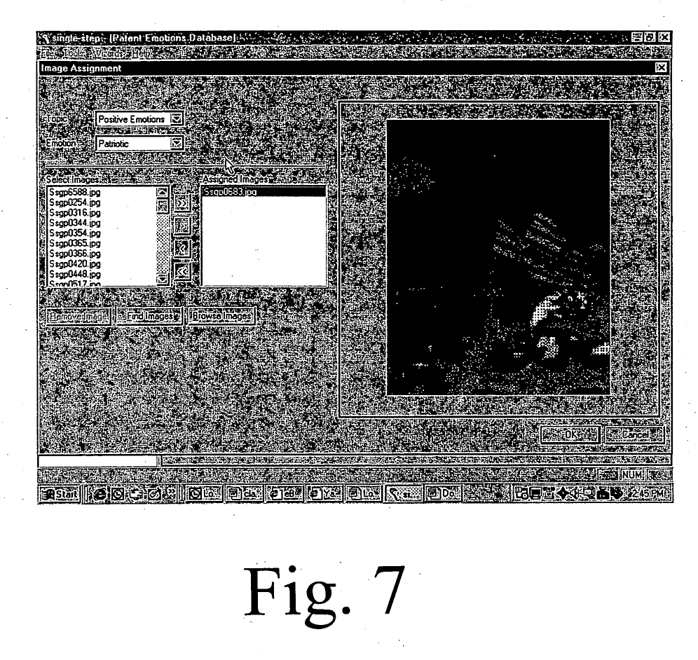

A description of a process for assigning media samples evoking a selected emotional state in the particular user to a corresponding electronically recallable set will now be described. Returning to FIG. 3, selection of either topic in the directory tree and subsequent selection of the “Maintain Images” button causes the screen shown in FIG. 7 to appear. Drop-down lists are provided for selection of the desired topic and emotional category, which in FIG. 7 are “Positive Emotions” and “Patriotic,” respectively. Media samples are then presented to the particular user one at a time. For the purposes of this detailed description, the media samples are presented in the form of a visual image. As discussed above, media samples appealing to other senses may be used in addition to or in lieu of visual images. For each image, the particular user provides an emotional response as to whether the image evokes a patriotic emotion. If the image (e.g., American flag as shown in FIG. 7) evokes a patriotic emotion in the particular user, the image is assigned to the emotional category/set by clicking the greater than “>” arrow. (The less than “<” arrow allows an image to be unselected. The signs “>>” and “<<” allow for selection and un-selection of all images.) If the image does not evoke a patriotic emotion in the particular user, the next image is presented to the particular user. This process is repeated until all images have been viewed or until a predetermined number of images have been assigned to the patriotic emotion. The selected emotion may then be changed using the drop-down lists, and the images may be viewed again in a similar manner to assign media samples to other emotional state categories.

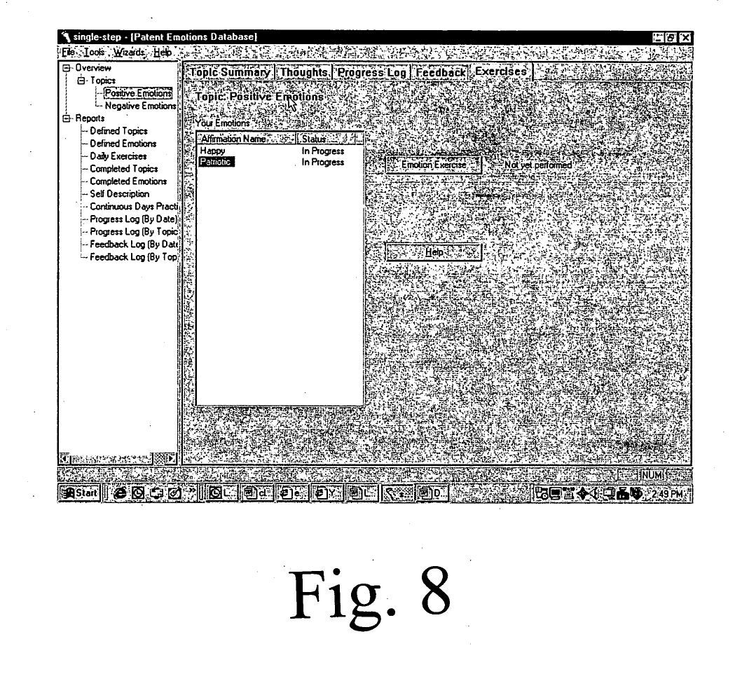

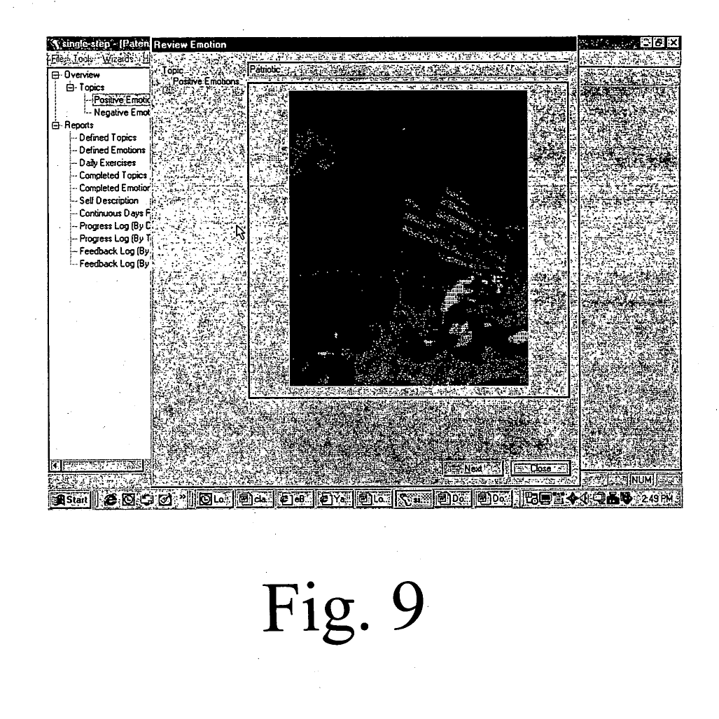

At a selected time to evoke the emotional state, the particular user is electronically presented with the media sample. Shown in FIG. 8, the “Emotion Exercise” button in the “Exercises” folder is selected. The particular user is then shown the selected image (e.g., American flag in FIG. 9) and, if desired, other images in the category until the corresponding emotional state is evoked in the user.

Referring back to FIG. 3, the program optionally further includes folders for “Thoughts,” “Progress Log,” and “Feedback.” The directory branch of FIG. 3 lists various reports that may be generated by the program. The example reports shown in FIG. 3 include the following: defined topics, defined emotions, defined exercises, completed topics, completed emotions, self description, continuous days practiced, progress logs by date or topic, and feedback logs by date or topic. These reports may assist the user in tracking progress or use of the program, but are not necessary to the successful use of the program.

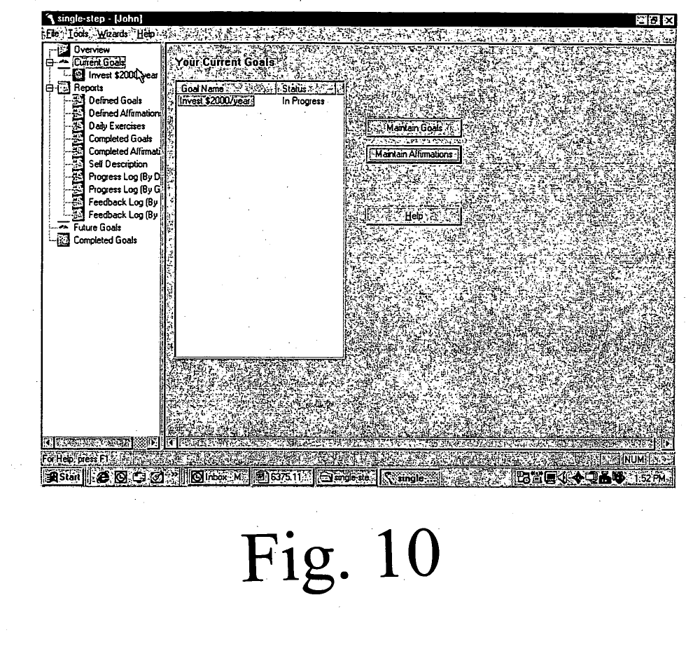

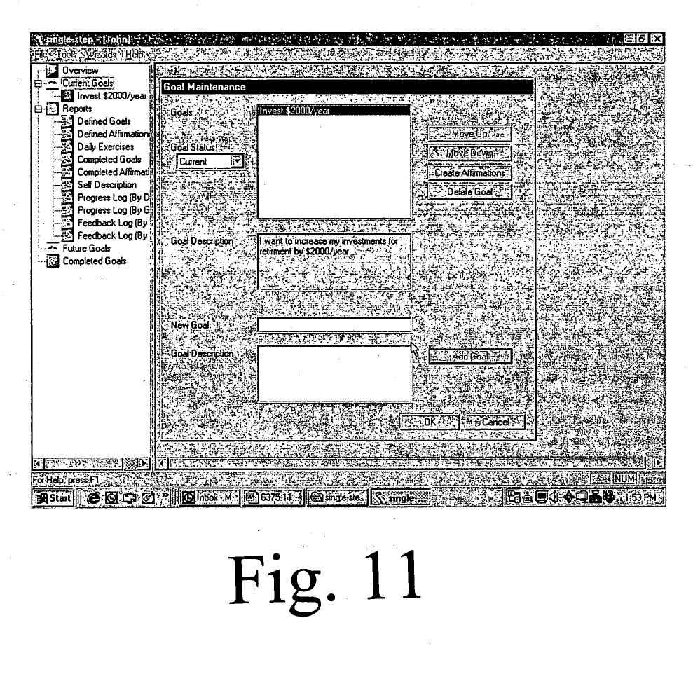

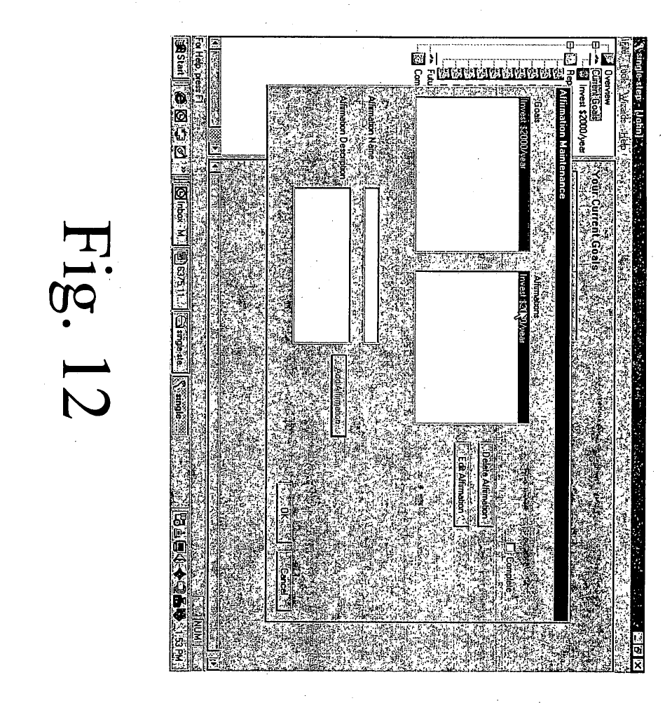

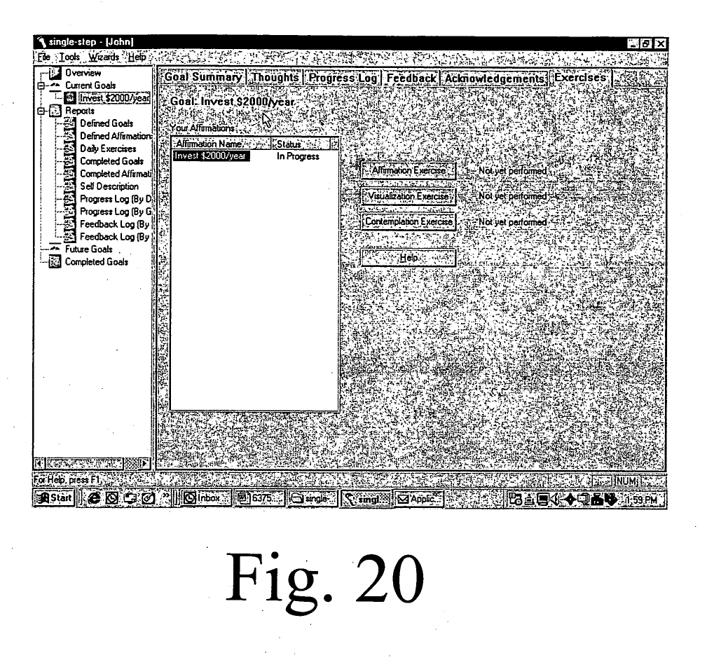

Turning now to FIGS. 10 through 22, user interface screen printouts of another program capable of carrying out methods of the present invention are shown. This second program is aimed towards assisting the user attain a goal successful. For the purposes of this description, and as shown in FIG. 10, the goal has been arbitraryily selected to comprise adherence to an investment strategy by which the user will invest $2000.00 per year. It should be understood that the program is capable of assisting in the realization of other related or unrelated goals. The particular goal to be achieved may be modified or deleted, or additional goals may be added, by selection of the “Maintain Goals” button in FIG. 10 and subsequent maintenance using the commands (e.g., “move up,” “move down,” “create affirmations,” “delete goal,” “add goal” shown in FIG. 11. Likewise, affirmations of each goal may be edited, added, or deleted by selection of the “Maintain Affirmations” button in FIG. 10 and subsequent maintenance using the commands (e.g., delete, edit, add) shown in FIG. 12.

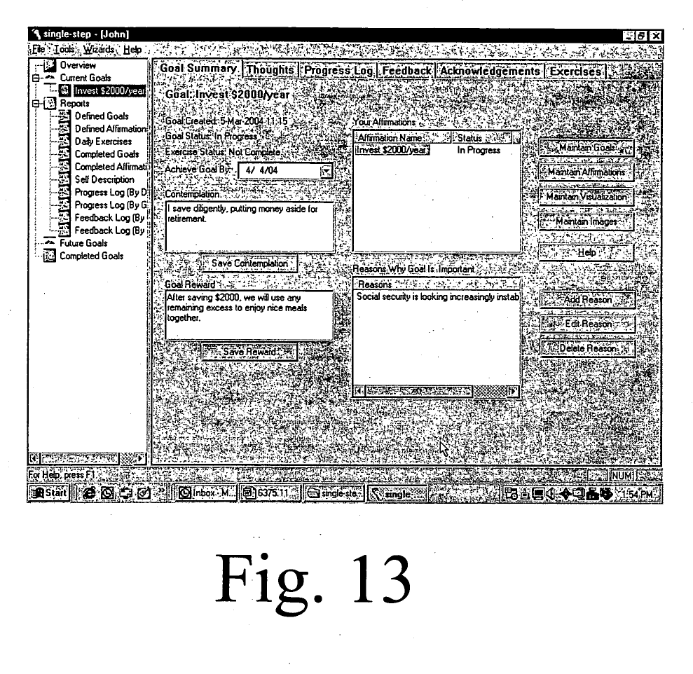

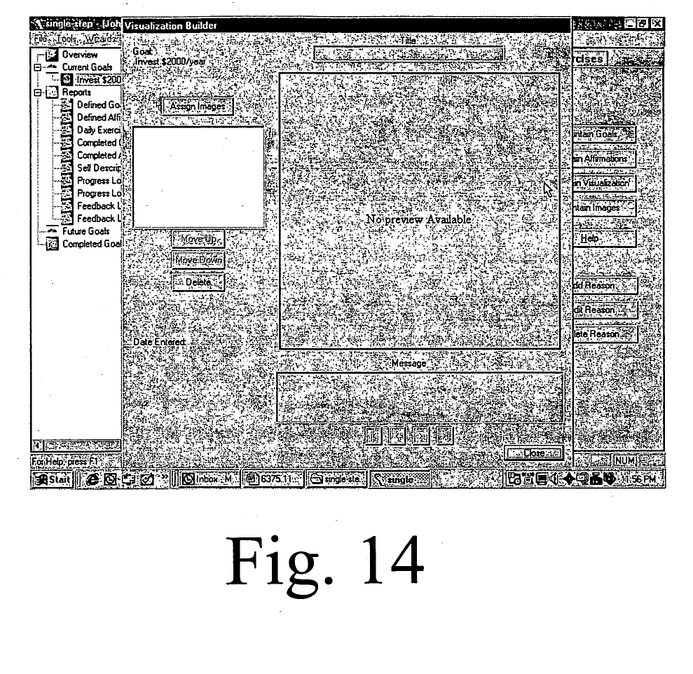

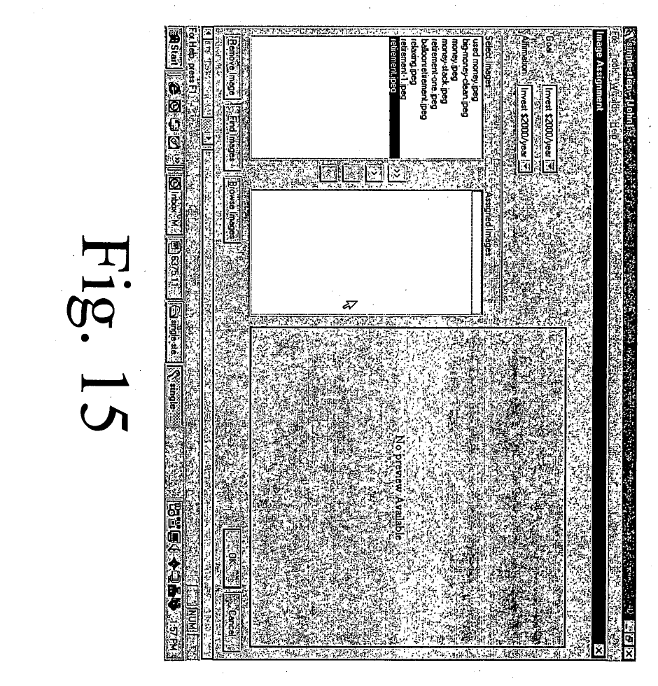

Selection of a goal from the directory tree (on the left side of FIGS. 10-12) will generate FIG. 13, which provides means for maintenance of the selected goal (e.g., “invest $2000/year”). The “Maintain Goals” and “Maintain Affirmations” buttons of FIG. 13 will essentially take the user to the screen printouts shown and previously described in FIGS. 11 and 12, respectively. The “Maintain Visualization” button of FIG. 13 directs the user to the screen of FIG. 14, where the particular user may assign media samples relating to the particular goal into a set, and permit future electronic recall of the media sample in the set at a selected time, such as in the case of a counseling session in which a particular emotional state is to be evoked in the user. The “Maintain Images” button of FIG. 13 directs the user to the screen of FIG. 15, where the particular user may assign media samples (e.g., images) to sets that may be recalled during exercises relating to the affirmation. Also provided on the screen shown in FIG. 13 are command buttons for adding, editing, and deleting reasons relating to why the goal is important to the particular user.

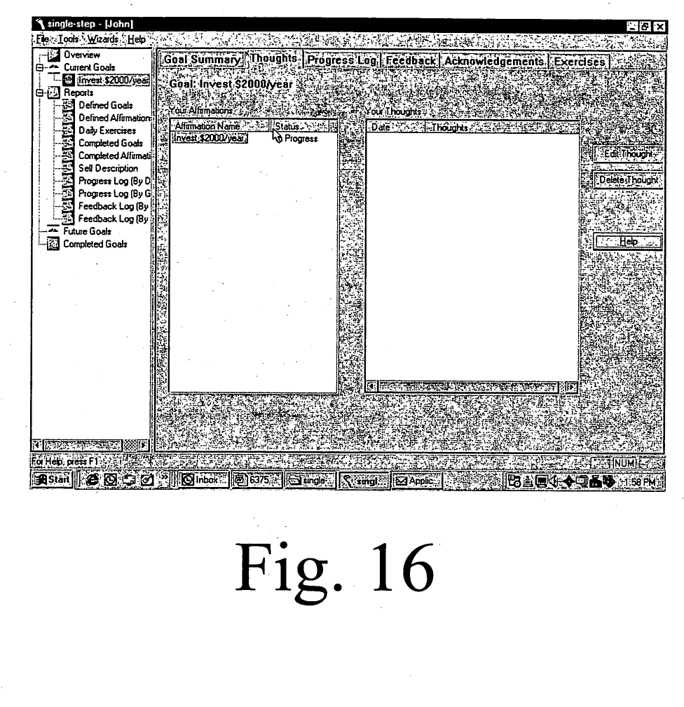

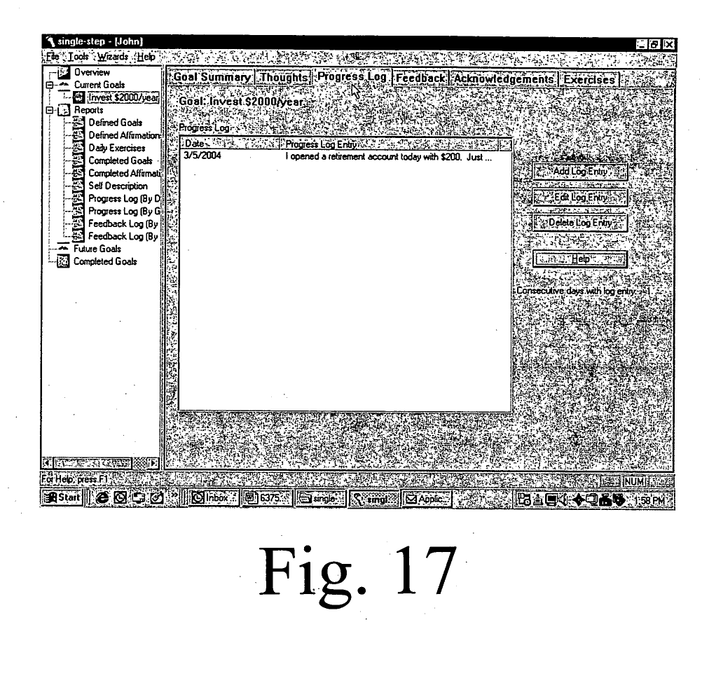

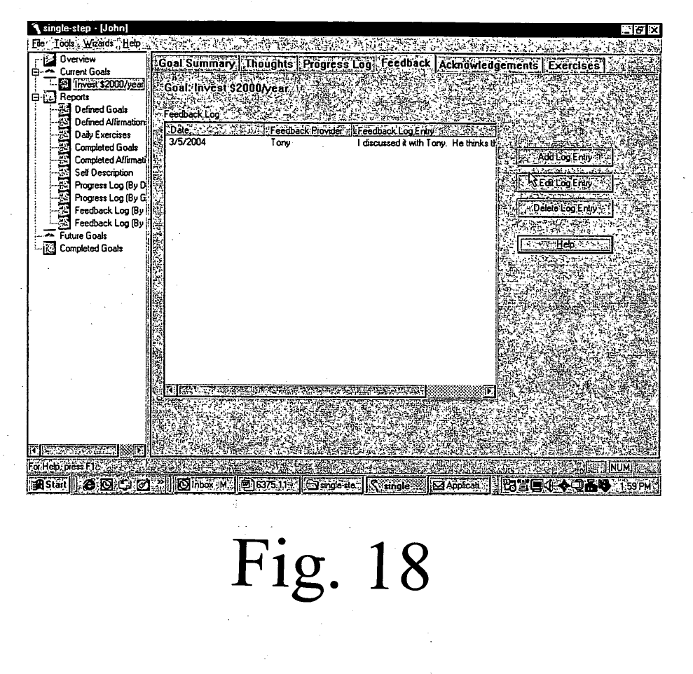

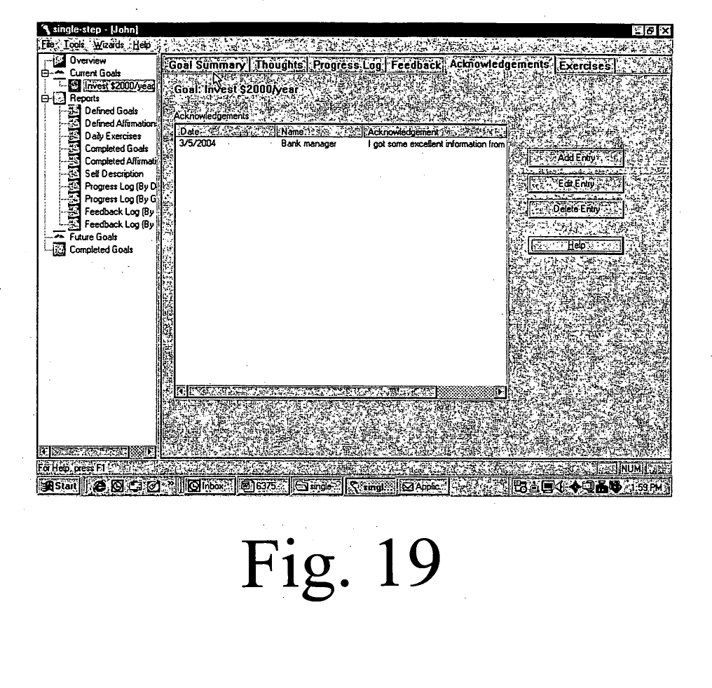

The computer program further provides folders relating to the following: thoughts (FIG. 16), in which the particular user may log his or her thoughts relating to the goal for future reference; progress log (FIG. 17), in which the particular user may log progress made in attainment of the goal; feedback (FIG. 18), in which the particular user may log any positive or negative comments that others have provided relating to his or her achievement of the goal; and acknowledgements (FIG. 19), in which the particular user may log names and associated information of persons who assisted in the course of seeking to achieve the goal.

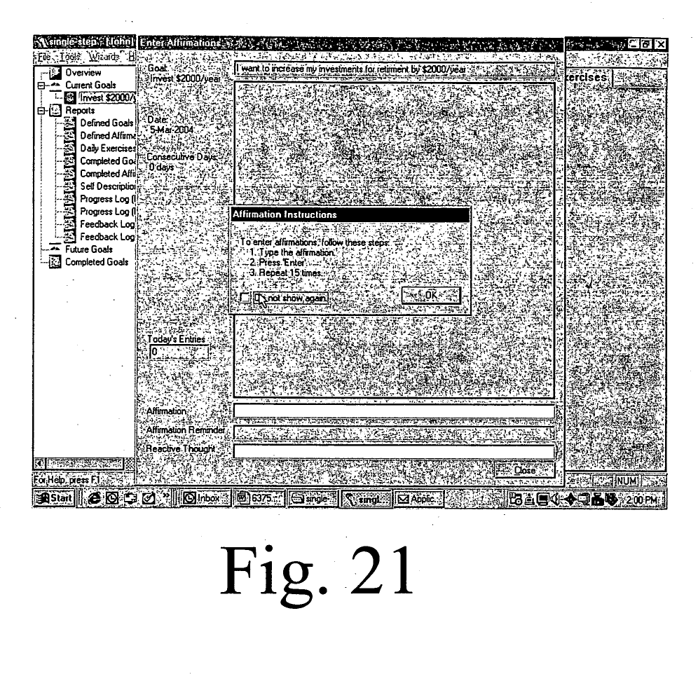

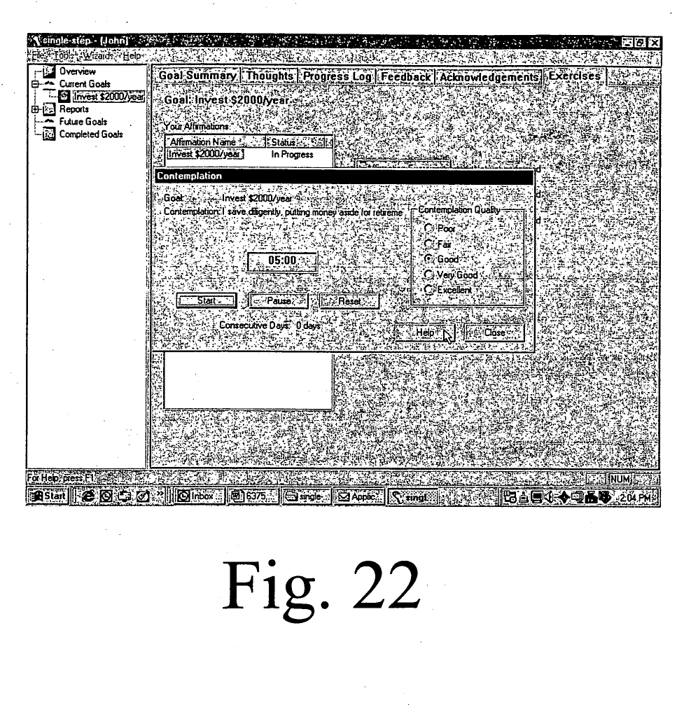

The rightmost folder provides exercises for the particular user. These exercises include an affirmation exercise, a visualization exercise, and a contemplation exercise. An example of an affirmation exercise is shown in FIG. 21, in which the particular user is instructed to type in an affirmation a preset (e.g., 16 in the figure) number of times. During performance of the exercise, the particular user is presented with a self-selected media sample, such as a visual image assigned in connection with FIG. 15. Visualization exercises generally involve the particular user reviewing one or more self-selected media samples of a set to evoke an emotion corresponding to the media samples. Visualization is particularly useful in goal achievement exercises and the like. The contemplation exercise is illustrated in FIG. 22, and in the illustrated embodiment comprises providing the particular user with instructions to mentally contemplate his or her goal for a predetermined amount of time, then providing the particular user with a means for recording the quality of the contemplation.

The foregoing detailed description of the certain preferred embodiments of the invention has been provided for the purpose of explaining the principles of the invention and its practical application, thereby enabling others skilled in the art to understand the invention for various embodiments and with various modifications as are suited to the particular use contemplated. This description is not intended to be exhaustive or to limit the invention to the precise embodiments disclosed. Modifications and equivalents will be apparent to practitioners skilled in this art and are encompassed within the spirit and scope of the appended claims.

1. A method of categorizing electronically recallable media, comprising:
(a) presenting a particular user with media samples possessing subject matter containing sensory stimuli;
(b) permitting the particular user to provide personal responses to the sensory stimuli of the media samples;
(c) determining from the personal responses whether the media samples evoke a selected emotional state in the particular user; and
(d) assigning media samples evoking the selected emotional state in the particular user to an electronically recallable set, the electronically recallable set being labeled with an emotional state identifier representative of the selected emotional state.

2. A method according to claim 1, wherein said step (a) comprises electronically presenting the particular user with a visual sample via a display unit.

3. A method according to claim 1, wherein said step (a) comprises electronically presenting the particular user with an audio sample via an output unit.

4. A method according to claim 1, wherein said step (a) comprises presenting the particular user with an olfactory sample.

5. A method according to claim 1, wherein said step (a) comprises presenting the particular user with at least two media samples selected from the group consisting of a visual sample, an audio sample, and an olfactory sample.

6. A method according to claim 1, further comprising storing the media samples in an electronic database.

7. A method according to claim 1, wherein said step (c) comprises the particular user determining whether the media samples evoke the selected emotional state.

8. A method according to claim 1, wherein said step (c) comprises an outside evaluator determining from the personal responses whether the media samples evoke the selected emotional state in the particular user.

9. A method according to claim 1, wherein said step (c) comprises determining whether the personal responses meet a predefined criterion for evoking the selected emotional state in the particular user.

10. A method according to claim 9, wherein the personal responses comprise physiological and/or physical responses of the particular user and the predefined criterion comprises predetermined physiological and/or physical levels, and wherein said method further comprises measuring and/or recording the physiological and/or physical responses via an external device.

11. A method according to claim 10, wherein the external device comprises a member selected from electroencephalograph (EEG) electrodes, positron emission tomography (PET), computerized axial tomography (CAT) scan, and magnetic resonance imaging (MRI).

12. A method according to claim 1, further comprising providing a computer program for categorizing the media samples into the electronically recallable set.

13. A method of categorizing electronically recallable media, comprising:
(a) presenting a particular user with media samples possessing subject matter containing sensory stimuli;
(b) permitting the particular user to provide personal responses to the sensory stimuli of the media samples;
(c) determining from the personal responses whether the media samples evoke one or more selected emotional states in the particular user; and
(d) electronically assigning the media samples to electronically recallable sets corresponding to the emotional states evoked in the particular user by the media samples, each of the electronically recallable sets being labeled with a respective emotional state identifier representative of the respective selected emotional state.

14. A method according to claim 13, wherein said step (a) comprises electronically presenting the particular user with a visual sample via a display unit.

15. A method according to claim 13, wherein said step (a) comprises electronically presenting the particular user with an audio sample via an output unit.

16. A method according to claim 13, wherein said step (a) comprises presenting the particular user with an olfactory sample.

17. A method according to claim 13, wherein said step (a) comprises presenting the particular user with at least two media samples selected from the group consisting of a visual sample, an audio sample, and an olfactory sample.

18. A method according to claim 13, further comprising storing the media samples in an electronic database.

19. A method according to claim 13, wherein said step (c) comprises the particular user determining whether the media samples evoke the selected emotional state.

20. A method according to claim 13, wherein said step (c) comprises an outside evaluator determining from the personal responses whether the media samples evoke the selected emotional state in the particular user.

21. A method according to claim 13, wherein said step (c) comprises determining whether the personal responses meet a predefined criterion for evoking the selected emotional state in the particular user.

22. A method according to claim 21, wherein the personal responses comprise physiological and/or physical responses of the particular user and the predefined criterion comprises predetermined physiological and/or physical levels, and wherein said method further comprises measuring and/or recording the physiological and/or physical responses via an external device.

23. A method according to claim 22, wherein the external device comprises a member selected from electroencephalograph (EEG) electrodes, positron emission tomography (PET), computerized axial tomography (CAT) scan, and magnetic resonance imaging (MRI).

24. A method according to claim 13, further comprising providing a computer program for categorizing the media samples into the electronically recallable sets.

25. A method of evoking an emotional state in a particular user through selective presentation of media samples, comprising:
(a) providing an electronically recallable set of self-selected media samples possessing subject matter containing sensory stimuli, the media samples being custom selected by a particular user for evoking a selected emotional state in the particular user; and
(b) electronically presenting the particular user with the media samples at a selected time to evoke the selected emotional state in the particular user.

26. A method according to claim 25, wherein said providing step (a) comprises:
(a)(i) presenting the particular user with the media samples;
(a)(ii) permitting the particular user to provide personal responses to the sensory stimuli of the media samples;
(a)(iii) determining from the personal responses whether the media samples evoke the selected emotional state in the particular user; and
(a)(iv) assigning media samples evoking the selected emotional state in the particular user to an electronically recallable set, the electronically recallable set being labeled with an emotional state identifier corresponding to the selected emotional state.

27. A method according to claim 26, further comprising selecting the electronically recallable set to effect the electronic presentation of the media samples.

28. A method according to claim 26, further comprising providing a computer program for categorizing the media samples into the electronically recallable set.

29. A method according to claim 25, wherein said providing comprises:
(a)(i) presenting the particular user with the media samples;
(a)(ii) permitting the particular user to provide personal responses to the sensory stimuli of the media samples;
(a)(iii) determining from the personal responses whether the media samples evoke one or more of the selected emotional states in the particular user; and
(a)(iv) electronically assigning the media samples to electronically recallable sets corresponding to the emotional states evoked in the particular user by the media samples

30. A method according to claim 29, wherein the electronically recallable sets are labeled with corresponding emotional state identifiers.

31. A method according to claim 29, wherein said step (a)(i) comprises electronically presenting the particular user with a visual sample via a display unit.

32. A method according to claim 29, wherein said step (a)(i) comprises electronically presenting the particular user with an audio sample via an output unit.

33. A method according to claim 29, wherein said step (a)(i) comprises presenting the particular user with an olfactory sample.

34. A method according to claim 29, wherein said step (a)(i) comprises presenting the particular user with at least two media samples selected from the group consisting of a visual sample, an audio sample, and an olfactory sample.

35. A method according to claim 29, further comprising storing the media samples in an electronic database.

36. A method according to claim 29, wherein said step (a)(iii) comprises the particular user determining whether the media samples evoke one of the selected emotional states in the particular user.

37. A method according to claim 29, wherein said step (a)(iii) comprises an outside evaluator determining from the personal responses of the user whether the media samples evoke one of the selected emotional states in the particular user.

38. A method according to claim 29, wherein said step (a)(iii) comprises determining whether the personal responses meet a predefined criterion for evoking the selected emotional state in the particular user.

39. A method according to claim 38, wherein the personal responses comprise physiological and/or physical responses of the particular user and the predefined criterion comprises predetermined physiological and/or physical levels, and wherein said method further comprises measuring and/or recording the physiological and/or physical responses via an external device.

40. A method according to claim 39, wherein the external device comprises a member selected from electroencephalograph (EEG) electrodes, positron emission tomography (PET), computerized axial tomography (CAT) scan, Galvanic Skin Resistance (GSR), and magnetic resonance imaging (MRI).

41. A method according to claim 29, wherein the selected time comprises a period immediately after the particular user has successfully completed a designated task.

42. A method according to claim 29, further comprising selecting the electronically recallable set to effect the electronic presentation of the media samples.

43. A method according to claim 29, further comprising providing a computer program for categorizing the media samples into the electronically recallable set.

44. A method according to claim 29, wherein the selected time comprises a period immediately after the particular user has exhibited positive behavior modification.

45. A method according to claim 29, wherein the selected time is during a psychological counseling session.

46. A method according to claim 29, wherein said electronically presenting step is repeated until the particular user reaches the selected emotional state.

47. A method according to claim 25, wherein the selected time comprises a period immediately after the particular user has successfully completed a designated task.

48. A method according to claim 25, wherein the selected time comprises a period immediately after the particular user has exhibited positive behavior modification.

49. A method according to claim 25, wherein the selected time is during a psychological counseling session.

50. A method according to claim 25, wherein said electronically presenting step is repeated until the particular user reaches the corresponding emotional state.

51. A computer usable medium for compiling electronically recallable sets of media samples customized for evoking corresponding emotional states in a particular user, the computer usable medium comprising:
(a) computer readable program code means for causing a computer to display a category entry field to a particular user via a display unit;
(b) computer readable program code means for causing the computer to receive an inputted electronically recallable category or categories into the category entry field, said electronically recallable category or categories each corresponding to a respective emotional state;
(c) computer readable program code means for presenting the particular user with a plurality of media samples, the media samples possessing subject matter containing sensory stimuli;
(d) computer readable program code means for permitting the media samples to be electronically assigned to the electronically recallable category or one or more of the electronically recallable categories based on the emotional states evoked in the particular user by the media samples; and
(e) computer readable program code means for permitting the particular user to electronically recall the media samples by the electronically recallable category or categories.

52. A system for compiling electronically recallable sets of media customized for evoking corresponding emotional states in a particular user, the system comprising:
(a) a computing device comprising the computer program;
(b) an input device;
(c) an output device;
(d) data storage; and
(e) said computer program for providing a particular user with a plurality of categories each corresponding to a respective emotional state, obtaining from the data storage media samples possessing subject matter containing sensory stimuli, presenting the media samples to the particular user via the output device and permitting the particular user to provide personal responses to the media samples, electronically assigning the media samples via the input device into one or more electronically recallable sets of the categories based on emotional states evoked by the media samples, and permitting selective electronic recall of the media samples.
